(19) United States
(12) Patent Application Publication
(10) Pub. No.: US 2013/0295518 A1
(43) Pub. Date: Nov. 7, 2013

(54) Apparatus and Method for Achieving a Head Up Posture for a 3-D Video Image for Operative Procedures in Dentistry

(71) Applicant: William S. Parker, Ann Arbor, MI (US)

(72) Inventor: William S. Parker, Ann Arbor, MI (US)

(21) Appl. No.: 13/853,153

(22) Filed: Mar. 29, 2013

Related U.S. Application Data
(60) Provisional application No. 61/617,461, filed on Mar. 29, 2012.

(51) Int. Cl. A61B 1/00; A61B 1/04; A61B 19/00; A61B 1/24

(52) U.S. Cl. CPC A61B 1/00048; A61B 1/24; A61B 1/04; A61B 1/00193; A61B 1/00009; A61B 1/0005; A61B 1/00149; A61B 19/5223
USPC 433/29; 433/215

(57) ABSTRACT

An apparatus and method to rigidly connect the imaging elements such as camera(s) and the monitor on a single arm, or a single arm element, which maintains the proper relationship between the camera and the monitor while simultaneously allowing the monitor height to be at the ideal viewing height for the dentist. While the imaging elements of the invention are rigidly connected on a single arm or arm element, there is provision for changing the tilt, on a horizontal axis, of the monitor, as well as its height, to accommodate differences in height among dentists. This rigid connection of the invention assures that the image of the operative site will always be presented in the most understandable frame of reference for the dentist.

This application is related to and claims priority to U.S. Provisional Patent application No. 61/617,461 filed Mar. 29, 2012. Application 61/617,461 is hereby incorporated by reference in its entirety.

### BACKGROUND

1. Field of the Invention

The present invention relates generally to performing dental work and more particularly to performing dentistry from a magnified video image in a comfortable head up posture.

2. Description of the Prior Art and Problem

Ten percent (10%) of the practicing dentists retire early; another twenty percent (20%) reduce their practice hours because of chronic neck and back pain and disabling neck injuries. The affected dentists suffer great economic losses which in aggregate are estimated to be $10 billion annually in the U.S. An individual dentist who retires early may lose 5-10 years of peak earning because of work related back and neck injury.

Dental surgery requires that precision and detailed work be performed in a poorly lit oral cavity. Recently composite restorative materials and bonded restorative techniques have become the standard of care and require improved visual acuity for success. These new materials and techniques have created the need for magnification in dentistry; and the use of magnifying loupes have become widespread.

Loupes provide the wearer with needed magnification however, the narrow field of view and the weight of the loupes makes their use uncomfortable and often add to a dentist's poor posture and likelihood of neck and back injury. These neck and back injuries are the most common cause of temporary and even permanent disability among dentists.

Operating Microscopes provide magnified images but the immobile, static nature of the eyepieces, combined with the awkward positions that must be assumed by the patient and the dentist, for proper viewing of many areas of dental anatomy, do not fully alleviate the poor posture or discomfort associated with doing dental procedures, and can exacerbate the discomfort.

Video cameras have demonstrated their ability to provide magnified images and excellent resolution of the oral anatomy, and systems incorporating them can also permit improved posture and comfort. A properly positioned video monitor can provide an ideal head up posture for the dentist, while performing dental procedures; thus eliminating or reducing the need to look directly into the patient's mouth with its associated awkward or uncomfortable posture.

U.S. Pat. No. 5,803,905 to Allred, U.S. Pat. No. 6,414,708 to Carmeli et al, U.S. Pat. No. 5,867,210 to Rod, and U.S. Pat. No. 7,443,417 B1 to Heinrich and published Patent Application 2006/0252004 A1 to Donahoo all disclose video viewing apparatus or methods which may be applied to dental procedures. Heinrich describes the need for a mirror image to provide an understandable frame of reference so that movement of dental instruments left to right in the mouth corresponds to movements from left to right across the video screen.

Single camera images of a dental operative site, as in Patent Application 2006/0252004 A1 by Donahoo, do not provide the dentist with depth perception, which is critical to efficient and accurate dental surgery, while Carmeli and Rod disclose stereoscopic (3D) imaging be various means.

The prior art also discloses the need for complex manipulation of the images with reversals from left to right and from top to bottom to provide an image with the proper orientation to give the operator an understandable frame of reference. Such image manipulation being dictated by the position and orientation (or perspective) of the video camera in relation to the normal observation position of the dentist, and the position of the video monitor. The image manipulations proposed as necessary by the prior art for an optimal image presentation are dependent upon understanding the position of four separate elements of the imaging process: 1) the dentist's head, or viewing perspective, 2) the patient's head, as the object of the image, 3) the position of the camera, and 4) the position of the monitor.

These manipulations when programmed or set-up manually require a complex spatial understanding by the dentist and also require extended training on the apparatus; and sometimes require difficult set-up prior to performing dental procedures. Such set-up may also need to be repeated on a single patient when the different areas of the oral anatomy must be viewed during dental surgery. In addition, a monitor which is remotely located and which may be rigidly affixed to the wall often is not and cannot be positioned correctly to present the proper image orientation to the dentist performing surgery. All these identified factors reduce the usefulness of the apparatus and inhibit its widespread use in dental surgeries.

When the set-up referenced above requires as much as two or three minutes and this set-up may be repeated two or three times for a given patient, the dentist's productivity suffers, the usefulness of the equipment is greatly compromised, and its use diminished.

For comfortable viewing and proper posture, the video monitor must be at the dentist's eye level. In order for the image on the monitor to be usefully and efficiently presented, all four elements referenced above must be positioned and maintained in the same vertical plane.

All examples of the prior art show cameras and monitors mounted on separate and independent articulated arms and further they show camera placements which often are not easily related to the dentist's viewing position, the dentist's head and the patient's head, or operative site. In short, the prior art requires that the camera and the monitor must be separately manipulated in three axis, and they must be further manipulated to bring their position into alignment with the vertical plane established by the dentist's and patient's head. All this manipulation is difficult and time consuming.

### SUMMARY OF THE INVENTION

The present invention relates to an apparatus and method to rigidly connect the imaging elements such as camera(s) and the monitor on a single arm, or a single arm element, which maintains the proper relationship between the camera and the monitor while simultaneously allowing the monitor height to be at the ideal viewing height for the dentist. While the imaging elements of the invention are rigidly connected on a single arm or arm element, there is provision for changing the tilt, on a horizontal axis, of the monitor, as well as its height, to accommodate differences in height among dentists. This rigid connection of the invention assures that the image of the operative site will always be presented in the most understandable frame of reference for the dentist.

It is an object of this invention to allow the dentist to have a 3-D view of the operative site by means of a stereo image produced by two cameras positioned in a field near to the operative site. It is an object of the invention to provide an image with intuitive depth perception similar to that which a dentist would have from direct visualization of the operative site.

It is further an object of this invention to use an optical magnifying means similar to the optics carrier of a surgical or dental microscope which is positioned in a field close to the patient's mouth and below the dentist's field of view to the monitor. When the camera is positioned near to the patient's mouth, for example, with an objective lens focal length of around 200-250 mm, and the rigid relationship between the camera and the monitor are established, the camera and the magnifying means do not obstruct the dentist's operative view of the monitor or the direct viewing of the patient's mouth.

It is further an object of this invention to a provide simple distance estimation from the objective lens of the optical apparatus and the mouth or dental tissue within the mouth, by means of two or more converging laser pointing beams. This facilitates the rapid movement of the apparatus while minimizing the disruption to the surgical procedures, and allows the movement of the apparatus to a correct position by either looking at the patient's mouth or alternatively looking at the video monitor. The converging laser beams may be activated by touch or a switch on the optics carrier or by a footpedal and are only expected to be utilized when the viewing position of the apparatus is re-positioned during surgical procedures.

### DESCRIPTION OF THE FIGURES

Attention is now directed at several drawings that illustrate features of the present invention.

FIG. 1 shows a drawing of a dentist treating a patient using an embodiment of the present invention.

FIG. 2 shows a drawing of the relationships of the imaging elements in FIG. 1

FIG. 3 shows a side view of the apparatus with the removable convergence calibration fixture in place for setting the convergence plane of the 3-D images at 250 mm.

FIG. 4 shows a side view of the apparatus of FIG. 3 showing convergence of the laser beams, and the detachable optics carrier module with cameras and the video processing package.

Several drawings and illustrations have been presented to aid in understanding the present invention. The scope of the present invention is not limited to what is shown in the figures.

### DESCRIPTION OF THE INVENTION

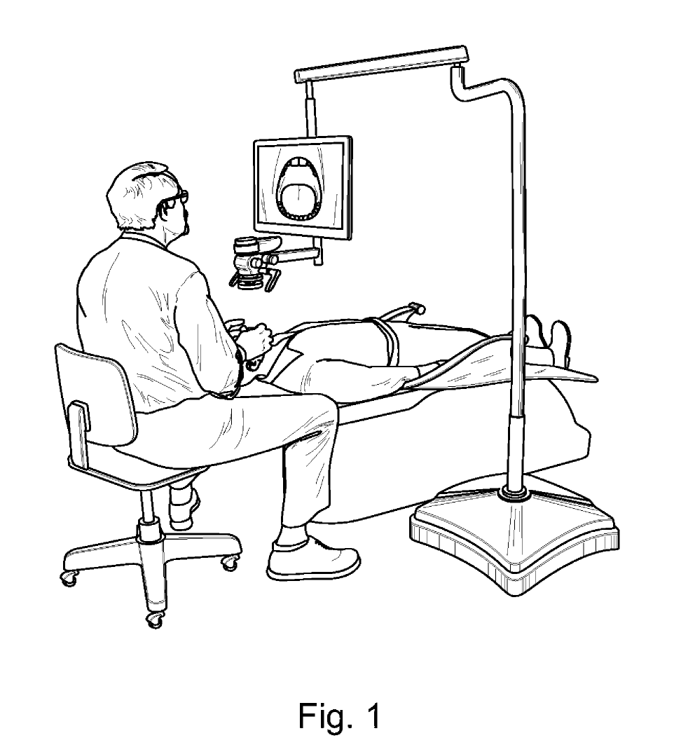

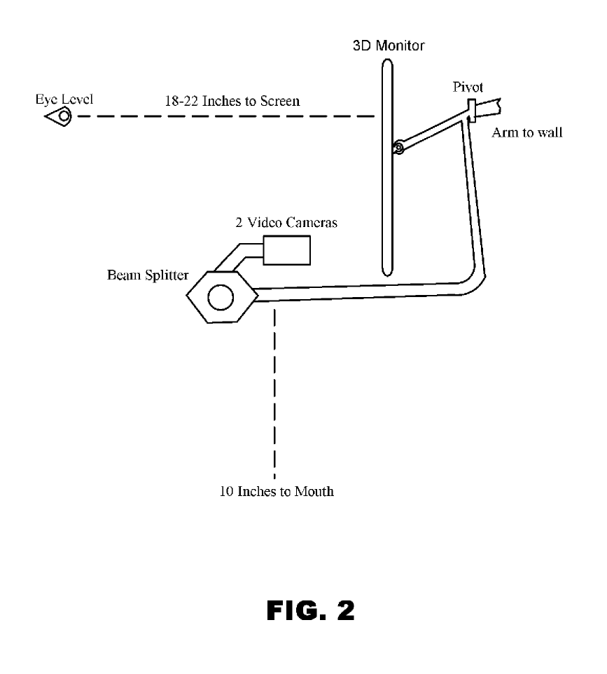

An object of this invention is to rigidly connect the imaging elements such as camera(s) and the monitor on a single arm, or a single arm element, which maintains the proper relationship between the camera and the monitor while simultaneously allowing the monitor height to be at the ideal viewing height for the dentist. This can be seen in FIG. 1 and is sketched in FIG. 2. While the imaging elements of the invention are rigidly connected on a single arm or arm element, there is provision for changing the tilt, on a horizontal axis, of the monitor, as well as its height, to accommodate differences in height among dentists. This rigid connection of the invention assures that the image of the operative site will always be presented in the most understandable frame of reference for the dentist.

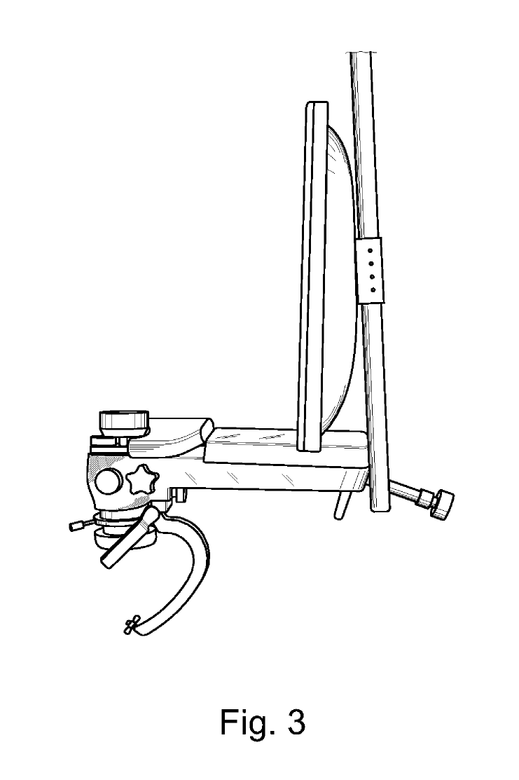

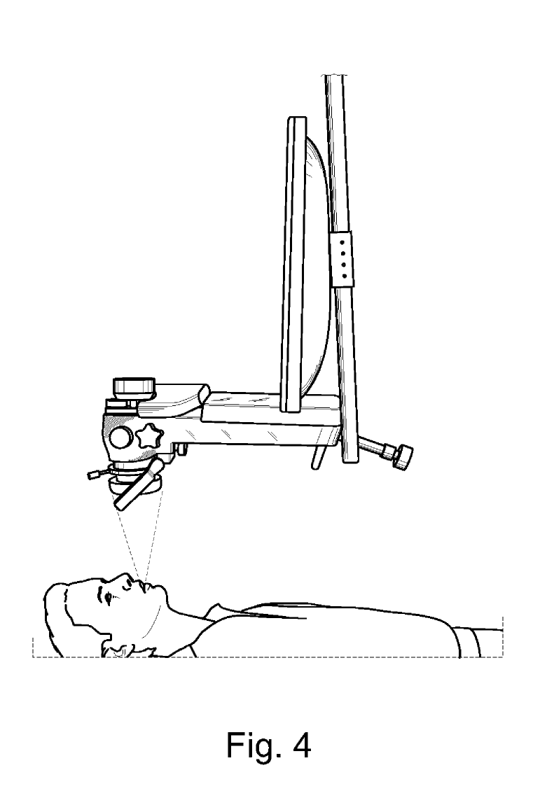

It is further an object of this invention to a provide simple distance estimation from the objective lens of the optical apparatus and the mouth or dental tissue within the mouth, by means of two or more converging laser pointing beams. This is shown in FIGS. 3-4. This will facilitate the rapid movement of the apparatus while minimizing the disruption to the surgical procedures, and allows the movement of the apparatus to a correct position by either looking at the patient's mouth or alternatively looking at the video monitor. The converging laser beams may be activated by touch or a switch on the optics carrier or by a footpedal and are only expected to be utilized when the viewing position of the apparatus is re-positioned during surgical procedures.

It is further an object of this invention to use an optical magnifying means similar to the optics carrier of a surgical or dental microscope which is positioned in a field close to the patient's mouth and below the dentist's field of view to the monitor. When the camera is positioned near to the patient's mouth, for example, with an objective lens focal length of around 200-250 mm, and the rigid relationship between the camera and the monitor are established, the camera and the magnifying means do not obstruct the dentist's operative view of the monitor or the direct viewing of the patient's mouth.

It is further an object of this invention to allow the dentist to have a 3-D view of the operative site by means of a stereo image produced by two cameras positioned in a field near to the operative site. It is an object of the invention to provide an image with intuitive depth perception similar to that which a dentist would have from direct visualization of the operative site.

When formulating 3-D images for comfortable viewing, it is beneficial to calibrate the focal plane of central objects in the 3-D display, and establish the convergence of the dual camera images at the plane of the video monitor. In this way, some of the video content in the 3-D monitor may appear in front of the monitor and some of the content will appear behind the surface plane of the monitor, while the central objects, the objects of the surgery, are presented for viewing at or about the plane of the monitor. Therefore, it is a further object of this invention to provide a removable calibrating jig or fixture which physically places a target, recognizable to the imaging software, at the same plane as the converging laser beams, and the same focal plane of the objective lens of the microscope-type optics carrier. In this way, the convergence plane of the dual images may be calibrated in a few seconds through a software program at the commencement of a surgical procedure, or even during a hiatus in the surgical procedure. The convergence calibration may be activated by the operator, or it may be automatically activated by the apparatus sensing the placement of the removable calibration fixture or jig.

It is further an object of this invention to provide the dentist with a 3-D image by means of a special commercially available 3-D monitor, such as a Hyundai W220S monitor or an LG 02342P monitor, designed to receive a single combined (composite) video signal which is a combination of the separate video signals from each of the two cameras. In a preferred embodiment, the video processing electronics interleaves the lines of the two video cameras so that the monitor means provides an image viewable with passive polarized glasses.

The separate video signals are generated by commercially available video cameras of 480 lines/frame, 720 lines/frame, or 1080 lines/frame; and 30 frames/second or 60 frames/second; and with optional interleaved image scanning or progressive image scanning technology. The separate video cameras may be selected from a wide range of commercially available video cameras or video sensor elements with the software and electronic processing being selectably or automatically capable of combining the image streams from a wide range of commercially available video cameras into a single suitable video stream for the 3-D monitor.

It is further an object of this invention to have the optical magnifying means provide the lighting for the operative site, similar to the lighting which would be provided by an operating, or surgical microscope. It is further the object of this invention to have multiple levels of magnification of the operative site. The levels of magnification encompass a range of 2× to 20× relative to the unmagnified view a dentist would receive with the unaided eye.

It is further an object of this invention to reliably place the monitor at a viewing distance which is considered ideal for the design of the monitor. The manufacturers of commercially available 3-D monitors typically specify the appropriate viewing angles and the ideal viewing distance for achieving the best 3-D image and 3-D effect. It is the object of this invention to use the geometry and dimensions between the imaging components of the single arm or single arm element to maintain the ideal viewing distance from the dentist to the monitor.

It is further an object of this invention to place the monitor for head up viewing at a location near enough to the dentist to allow for monitors which are smaller and less intrusive than the monitors shown in the prior art. Since the monitors shown in the prior art are mounted on separate articulated arms, it is often necessary that the monitors be placed 30″ to 48″, or 60″ or more from the viewing position of the dentist. In some cases, the monitors are even wall mounted at greater distances. This necessarily requires larger, more intrusive monitors to maintain a desired level of magnification. It is an object of this invention to be able to position the monitor close enough to the dentist so that monitor sizes from 11″ to 24″ can provide the necessary image magnification while being much less obtrusive.

It is further an object of this invention to place a second smaller monitor with a view convenient to a dental assistant helping with the dental surgery. It is anticipated that placing a 3-D monitor at the ideal viewing position for the dentist necessarily makes that monitor difficult to see for the dental assistant, who is typically positioned facing the patient about 90 degrees clockwise from of the dentist. A small 2-D or 3-D monitor movably positioned approximately perpendicular to the dentist's larger monitor will permit the dental assistant to view the procedures in the oral cavity and more easily assist the dentist.

It is further an object of this invention to place the video processing unit on the same arm or arm element to eliminate most of the costly and cumbersome video cables between the cameras, the processor, and the monitor. The electronics of the invention are small and light and can be included on the same arm as the imaging elements. In this configuration it is also possible for the optics carrier, the camera elements and the video processor to be a detachable module which may be moved from one dental operatory to another. When the detachable module is detached and the 3-D system is not in use in a particular operatory, the monitor and the positioning arm remain in the operatory and are available for the display of radiographic images or computerized patient information.

The present invention provides a method of performing dental work and an apparatus which provides a magnified 3-D video image of the oral cavity and which rigidly maintains the ideal relationship between the cameras and the monitor so that the image on the monitor, is always in an understandable frame of reference properly oriented to the dentist.

The video camera head of the invention includes a lighting assembly for illuminating the oral cavity, and the camera head and optical magnification assembly is positioned in a field near to the patient's oral cavity. With the cameras and optical assembly of the invention placed in a field near to the patient's oral cavity, the camera and optical assembly is below the field of view to the monitor, and dentists view of the monitor is not obstructed.

The invention also includes software and electronics, on the optics carrier arm, for producing a single combined video image stream (a 3-D composite video stream), viewable on a 3-D monitor; with the combined video stream being produced by processing the images from two cameras into a 3-D image suitable for presentation on a 3-D monitor.

The present invention also incorporates approximate dimensions and geometry in the rigid arm or arm element which permit the 3-D monitor to be positioned at an ideal distance for viewing, according to the monitor manufacturer's specifications; and in a comfortable head up position for the dentist to view the magnified 3-D images.

The present invention incorporates magnifying optics and a spatial relationship to the monitor which deliver magnifications suitable for general dental procedures, dental surgery, restorative dentistry, cosmetic dentistry and endodontic dental procedures.

The present invention also incorporates converging laser beams for rapid positioning of the apparatus over the operative field and a means of calibrating the convergence of the dual video images for comfortable 3-D viewing.

The present invention also incorporates the electronic video processing package on the optics carrier to eliminate most cabling and to permit the 3-D elements to be detachable as a module for movement from one dental operatory to another.

An example of the basic technology of the video processing unit can be found in application number PCT/US11/030,471. This technology embodies the real-time processing of video images from two video cameras for presentation on a 3-D monitor.

Several descriptions and illustrations have been provided to aid in understanding the present invention. One with skill in the art will realize that numerous changes and variations may be made without departing from the spirit of the invention. Each of these changes and variations is within the scope of the present invention.

1. A real-time 3-dimensional image apparatus for achieving a head up posture while performing a dental procedure, comprising:
two video cameras with associated magnification optics;
a stereoscopic monitor receiving images from said video cameras;
wherein, said apparatus is adapted to maintain a vertically planar relationship among said video cameras, said stereoscopic monitor, a first point in space proximate to the location of a patient's mouth during said dental procedure and a second point in space proximate to the location of a dentist's head during said dental procedure.

2. The apparatus of claim 1 wherein said apparatus is adapted to allow said stereoscopic monitor to be moved up or down or tilted to accommodate height differences among dentists while maintaining said stereoscopic monitor in said vertically planar relationship.

3. The apparatus of claim 1 further comprising two converging laser beams for locating an object of surgery at the center of the visual field of said cameras, wherein said laser beams converge at the focal length of said magnification optics.

4. The apparatus of claim 1 further comprising a removable fixture for calibrating a 3-dimensional plane of convergence for the two video cameras at the focal plane of the magnification optics.

5. The apparatus of claim 1 further comprising a second monitor positioned at ninety degrees to the stereoscopic monitor for viewing by a dental assistant.

6. The apparatus of claim 1 further comprising a removable module containing the video cameras, magnification optics and video processing elements.

7. The apparatus of claim 1 which utilizes a stereoscopic monitor which displays alternate lines from each of the two video cameras in an interleaved fashion, and which utilizes passive polarized glasses for viewing the real-time 3-dimensional image.

8. The apparatus of claim 1 which utilizes a auto-stereoscopic monitor which displays alternate lines from each of the two video cameras in an interleaved fashion, and which does not require special glasses for viewing the real-time 3-dimensional image.

9. The apparatus of claim 1 further comprising a surgical microscope, wherein said stereoscopic monitor displays images from said surgical microscope.

10. The apparatus of claim 1 wherein said video camera, said monitor, said magnification optics and video processing electronics are on a single removable arm which is adapted to position the monitor approximately 20″ distance from said first point in space, and which also enables the direct viewing distance from said first point in space to said second point in space to be at approximately 20″.

11. A real-time image apparatus for achieving a head up posture while performing a dental procedure, comprising:
a three-dimensional monitor adapted to receive real time images from a pair of video cameras focused to a first point in space proximate to the location of a patient's mouth during said dental procedure;
a removable arm holding said video cameras and said monitor, said arm being adapted to position said monitor approximately 20 inches horizontally from a second point in space proximate to the location of a dentist's head during said dental procedure;
said apparatus maintaining a vertical planar relationship between said video cameras, said monitor, said first point in space and said second point in space.

12. The apparatus of claim 11 wherein said apparatus is adapted to allow said three-dimensional monitor to be moved up or down or tilted to accommodate height differences among dentists while maintaining said three-dimensional monitor in said vertically planar relationship.

13. The apparatus of claim 11 further comprising two converging laser beams for locating an object of surgery at the center of the visual field of said cameras, wherein said laser beams converge at said first point in space.

14. The apparatus of claim 11 further comprising a removable fixture for calibrating a three-dimensional plane of convergence for the video camera pair.

15. The apparatus of claim 11 further comprising a second monitor positioned at ninety degrees to the three-dimensional monitor for viewing by a dental assistant.

16. (canceled)

17. The apparatus of claim 1 further comprising a removable module containing the video camera pair, magnification optics and video processing elements.

18. A method of providing a real-time 3D video image to a dentist during a dental procedure comprising:
mounting a video camera pair and a three-dimensional monitor on a movable arm so that the video camera pair, the three-dimensional monitor, a first point in space at the location of a patient's mouth during said dental procedure and a second point in space at the location of a dentist's head during said dental procedure have a vertical planar relationship;
also mounting said three-dimensional monitor approximately twenty inches horizontally from said second point in space.

19. The method of claim 18 further comprising providing two converging laser beams for locating an object at the center of the visual field of said cameras, wherein said laser beams converge at said first point in space.

20. The method of claim 18 wherein the three-dimensional monitor comprises a stereoscopic monitor which displays alternate lines from each video camera in an interleaved fashion, and which utilizes passive polarized glasses for viewing a real-time 3-dimensional image from said video camera pair.

21. The method of claim 18 wherein the three-dimensional monitor comprises a an auto-stereoscopic monitor which displays alternate lines from each video camera in an interleaved fashion, and which does not require special glasses for viewing a real-time 3-dimensional image from said video camera pair.
